SEQUENCE LISTING (2 sequences)

SEQ ID NO: 1 - DNA, 1200 bp, Corynebacterium glutamicum
  CDS (200)..(1006)
atcgcgatccgtcagatggtcttcctaccactgacctacgaccaccaggttgtagatggcgcagatgctggtcgcttcctgaccaccatcaaggaccgccttgagaccgctaacttcgaaggcgatctgcagctctaagatctctgcaagttaaaaccgccactcccctttcactggggagtggcggttttgtcgtttcatgcatgcagtgtgtgacttatcaaccttgttagggctagggtggatatctatcatgactgcaccaagagatccttttttccccgcagatctttctatccgcgcgtctgcagagcccattgaaattcagcggttgggtttgatcgattatcaagaggcctgggattatcaagcagagcttgctacccgtagggctaatgatgaaatccctgatcagctgcttattttggagcacccgtcggtgtataccgcaggtaagcgcacccagccggaagatcttcccaccaacggactgccggtgatcaatgctgatcgtggtggtcgcatcacgtggcatggtcctggccaattggtgatctatccgatcatcaaattagccgatccgatcgatgtggttgattacgtaagacgcctcgaggaagcgctcatccaagttgtcggcgatatgggtgttgccggcgctgggcgcattgatgggcgttcgggtgtgtgggtgccagctcatgatggttgggtggacagcaaggttgcggccatcggcattcgaataactcgtggtgttgcaatgcacggtgtggccatcaactgcaacaacacgttggatttctatgagcacatcattccgtgtggcattgctgatgcaggcttgagcacactctcgagggaactgaaaagggacgtttcagttgaggaattagtcgagccatcgatccgcgcattggatgatgctttggctggtcggctggttgtttctgatcattctttcggcagcgcgcccgacccaactaagaatctccctaaacgggggtagtacgaggaattttgtcggtggggcgcctcgttgaagcgaagtagagccgattgcagaatcggcggaatgagacgtcgaaaagcgtttaagctttccctaaaaatatcactaactcgaaagatgtaaggttgcatttgtgactatcgcacctgaaggacgacgactgctacgcgtcgaagctcgaaactcag

SEQ ID NO: 2 - protein, 269 aa, Corynebacterium glutamicum
MHAVCDLSTLLGLGWISIMTAPRDPFFPADLSIRASAEPIEIQRLGLIDYQEAWDYQAELATRRANDEIPDQLLILEHPSVYTAGKRTQPEDLPTNGLPVINADRGGRITWHGPGQLVIYPIIKLADPIDVVDYVRRLEEALIQVVGDMGVAGAGRIDGRSGVWVPAHDGWVDSKVAAIGIRITRGVAMHGVAINCNNTLDFYEHIIPCGIADAGLSTLSRELKRDVSVEELVEPSIRALDDALAGRLVVSDHSFGSAPDPTKNLPKRG

(19) United States
(12) Patent Application Publication
(10) Pub. No.: US 2002/0031809 A1
(43) Pub. Date: Mar. 14, 2002

(54) Nucleotide sequences coding for the lipB gene

(75) Inventors: Bettina Moeckel, Duesseldorf (DE); Walter Pfefferle, Halle (DE); Michael Buchholz, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 09/942,572

(22) Filed: Aug. 31, 2001

(30) Foreign Application Priority Data
Aug. 31, 2000 (DE) 100 42 739.1

(51) Int. Cl. C12P 13/04; C12Q 1/68; C07H 21/04; C12N 1/21

(52) U.S. Cl. 435/106; 435/6; 435/252.3; 435/320.1; 536/23.2

(57) ABSTRACT

The invention relates to polynucleotides corresponding to the lipB gene and which encode a lipoprotein ligase B, methods of producing L-amino acids, and methods of screening for polynucleotides which encode proteins having lipoprotein ligase B activity.

### CROSS-REFERENCE TO RELATED APPLICATIONS

The present application claims priority to the German Application No. DE 10042739.1 filed Aug. 31, 2000; the entire contents of which are incorporated herein by reference.

### BACKGROUND OF THE INVENTION

1. Field of the Invention

The invention provides nucleotide sequences from Coryneform bacteria which code for the lipB gene and a process for the fermentative preparation of amino acids, in particular L lysine, by attenuation of the lipB gene. The lipB gene codes for lipoprotein ligase B.

2. Discussion of the Background

L-amino acids, particularly L-lysine, are used in human medicine, in the pharmaceutical industry, in the foodstuffs industry, and, in particular, in animal nutrition.

It is known that amino acids are prepared by fermentation from strains of Coryneform bacteria, in particular Corynebacterium glutamicum. Due to its great importance, attempts are constantly being made to improve the preparation process. Improvements to the process may concerns measures relating to fermentation, for example, stirring and oxygen supply, or the composition of the culture media, such as, the sugar concentration during fermentation, or isolating the product form by ion exchange chromatography or the intrinsic output properties of the microorganism itself.

The output properties of these microorganisms are improved by employing methods of mutagenesis, selection, and mutant selection. These methods yield strains that produce amino acids, such as L-lysine, and are resistant to antimetabolites or are auxotrophic for metabolites important for regulation.

For a number of years, methods of recombinant DNA engineering have also been used to improve L-amino acid-producing strains of Corynebacterium. However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that attenuated expression of the lipB gene encoding the lipoprotein ligase B would improve L-amino acid yields.

### SUMMARY OF THE INVENTION

An object of the present invention is to provide novel measures for the improved production of L-amino acids or amino acids, where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-tryptophane, L-arginine, and in particular L-lysine and the salts (monohydrochloride or sulfate) thereof.

One object of the present invention is providing a novel process for improving the fermentative production of said L-amino acids, particularly L-lysine. Such a process includes enhanced bacteria, preferably enhanced Coryneform bacteria, which express attenuated amounts of the lipoprotein ligase B, which is encoded by lipB gene.

Thus, another object of the present invention is providing such a bacterium, which expresses an attenuated amount of lipoprotein ligase B or gene products of the lipB gene.

Another object of the present invention is providing a bacterium, preferably a Coryneform bacterium, which expresses a polypeptide that has attenuated lipoprotein ligase B activity.

Another object of the invention is to provide a nucleotide sequence encoding a polypeptide which has lipoprotein ligase B protein sequence. One embodiment of such a sequence is the nucleotide sequence of SEQ ID NO: 1.

A further object of the invention is a method of making lipoprotein ligase B or an isolated polypeptide having a lipoprotein ligase B activity, as well as use of such isolated polypeptides in the production of amino acids. One embodiment of such a polypeptide is the polypeptide having the amino acid sequence of SEQ ID NO: 2.

Other objects of the invention include methods of detecting nucleic acid sequences homologous to SEQ ID NO: 1, particularly nucleic acid sequences encoding polypeptides that have lipoprotein ligase B activity, and methods of making nucleic acids encoding such polypeptides.

The above objects highlight certain aspects of the invention. Additional objects, aspects and embodiments of the invention are found in the following detailed description of the invention.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

The invention provides a polynucleotide isolated from Coryneform bacteria containing a polynucleotide sequence coding for the lipB gene, selected from the group

a) a polynucleotide that is at least 70% identical to a polynucleotide that codes for a polypeptide containing the amino acid sequence SEQ ID No. 2,

b) a polynucleotide that codes for a polypeptide containing an amino acid sequence that is at least 70% identical to the amino acid sequence SEQ ID No. 2,

c) a polynucleotide that is complementary to the polynucleotides of a) or b), and

d) a polynucleotide containing a sequence of at least 15 nucleotides from the polynucleotide sequence of a), b) or c),

wherein the polypeptide preferably has the activity of lipoprotein ligase B.

The invention also provides the above-mentioned polynucleotide, preferably being a replicable DNA containing:

(i) the nucleotide sequence shown in SEQ ID No.1 or

(ii) at least one sequence that corresponds to sequence (i) within the degeneracy of the genetic code, or

(iii) at least one sequence that hybridizes with the sequences that are complementary to sequences (i) or (ii), and optionally

(iv) sense mutations in (i) that are neutral in terms of function.

The invention also provides:

a replicable DNA containing the nucleotide sequence shown in SEQ ID No.1;

a polynucleotide that codes for a polypeptide containing the amino acid sequence shown in SEQ ID No. 2;

a vector containing parts of the polynucleotide shown in SEQ ID No.1 that contains at least 15 consecutive nucleotides of said polynucleotide

and Coryneform bacteria that contains the vector carrying the lipB gene or in which the lipB gene is attenuated, in particular by an insertion or deletion.

The invention also provides polynucleotides consisting substantially of a polynucleotide sequence which are obtainable by screening by means of hybridization, of a Coryneform gene library containing the complete gene having the polynucleotide sequence shown in SEQ ID No.1, using a probe containing the sequence of said polynucleotide according to SEQ ID No.1 or a fragment thereof, and isolating said polynucleotide sequence.

Polynucleotide sequences according to the invention are suitable as hybridization probes for RNA, cDNA and DNA, in order to isolate nucleic acids or polynucleotides or full-length genes that code for lipoprotein ligase B or in order to isolate those nucleic acids and polynucleotides or genes that exhibit a high similarity with the sequence of the lipB gene.

The DNA of genes that code for the lipoprotein ligase B can be prepared with the PCR by using the polynucleotides according to the invention as primers.

Those oligonucleotides acting as probes or primers contain at least 30, preferably at least 20, most preferably at least 15 consecutive nucleotides. Also suitable are oligonucleotides that have a length of at least 40 or 50 consecutive nucleotides.

“Isolated” means removed from its natural surroundings.

“Polynucleotide” generally refers to polyribonucleotides and poldeoxyribonucleotides. The RNA or DNA may be modified or unmodified.

Polynucleotides according to the invention include a polynucleotide shown in SEQ ID No. 1 or a fragment prepared therefrom and also those that are at least 70%, preferably at least 80%, and most preferably at least 90% to 95% identical to the polynucleotide according to SEQ ID No. 1 or a fragment prepared therefrom.

“Polypeptides” are understood to be peptides or proteins that contain two or more amino acids linked via peptide bonds.

Polypeptides according to the invention include a polypeptide shown in SEQ ID No. 2, particularly those with the biological activity of lipoprotein ligase B, and also those that are at least 70%, preferably at least 80%, and most preferably at least 90% to 95% identical to the polypeptide in accordance with SEQ ID No. 2 and exhibit the mentioned activity.

The invention also provides a process for the production of amino acids, in particular L-lysine, using Coryneform bacteria that, in particular, already produce amino acids and in which the nucleotide sequences coding for the lipB gene are attenuated, in particular switched off or expressed at a low level.

The term “attenuation” in this connection describes the reduction or exclusion of the intracellular activity of one or more enzymes (proteins) in a microorganism that are coded by the corresponding DNA, by, for example, using a weak promoter or a gene or allele that codes for a corresponding enzyme with a low activity or by inactivating the corresponding gene or enzyme (protein) and optionally by combining those measures. As a result of attenuation, the activity or concentration of the corresponding protein is, in general, reduced to 0 to 50%, 0 to 25%, 0 to 10%, or 0 to 5% of the wild-type protein activity or concentration.

Microorganisms provided by the present invention may produce L-amino acids, in particular L-lysine, from glucose, saccharose, lactose, fructose, maltose, molasses, starch, cellulose or from glycerin and ethanol. These microorganisms may be representatives of Coryneform bacteria, in particular of the genus Corynebacterium. Corynebacterium glutamicum of this genus garners special mention since it is well known to those skilled in the art for its ability to produce L-amino acids.

Suitable strains of the genus Corynebacterium, particularly of the species Corynebacterium glutamicum (C. glutamicum), are especially the known wild strains

Corynebacterium glutamicum ATCC13032

Corynebacterium acetoglutamicum ATCC15806

Corynebacterium acetoacidophilum ATCC 13870

Corynebacterium melassecola ATCC17965

Corynebacterium thermoaminogenes FERM BP-1539

Brevibacterium flavum ATCC 14067

Brevibacterium lactofermentum ATCC13869 and

Brevibacterium divaricatum ATCC14020

or L-amino acid-producing mutants or strains prepared therefrom, for example, the L-lysine-producing strains

Corynebacterium glutamicum FERM-P 1709

Corynebacterium glutamicum FERM-P 6463

Corynebacterium glutamicum FERM-P 6464

Corynebacterium glutamicum DM58-1

Corynebacterium glutamicum DG52-5

Corynebacterium glutamicum DSM 5715

Corynebacterium glutamicum DSM 12866

Brevibacterium flavum FERM-P 1708 and

Brevibacterium lactofermentum FERM-P 1712

Preferably, a bacterial strain with attenuated expression of a lipB gene that encodes a polypeptide with lipoprotein ligase B activity will improve amino acid yield at least 1%.

The inventors have succeeded in isolating the lipB gene from C. glutamicum that codes for lipoprotein ligase B.

To isolate the lipB gene, or also other genes, from C. glutamicum, a gene library of that microorganism is prepared in Escherichia coli (E. coli). The preparation of gene libraries is described in generally known textbooks and manuals. For example, the textbook by Winnacker: Gene und Klone, Eine Einfuhrung in die Gentechnologie (Verlag Chemie, Weinheim, Germany, 1990), or the manual by Sambrook et al.: Molecular Cloning, A Laboratory Manual (Cold Spring Harbor Laboratory Press, 1989). A well-known gene library is that of E. coli K-12 strain W3110, which has been prepared by Kohara et al. (Cell 50, 495-508 (1987)) in λ vectors. Bathe et al. (Molecular and General Genetics, 252:255-265, 1996) describe a gene library from C. glutamicum ATCC13032, which was prepared with the aid of the cosmid vector SuperCos I (Wahl et al., 1987, Proceedings of the National Academy of Sciences USA, 84:2160-2164) in E. coli K-12 strain NM554 (Raleigh et al., 1988, Nucleic Acids Research 16:1563-1575). Börmann et al. (Molecular Microbiology 6(3), 317-326 (1992)) describe a gene library from C. glutamicum ATCC13032 using the cosmid pHC79 (Hohn and Collins, 1980, Gene 11, 291-298).

It is possible to use plasmids, such as pBR322 (Bolivar, 1979, Life Sciences, 25, 807-818) or pUC9 (Vieira et al., 1982, Gene, 19:259-268) to produce a gene library from C. glutamicum in E. coli. Suitable hosts are particularly E. coli strains that are restriction and recombination deficient such as the strain DH5α: (Jeffrey H. Miller: “A Short Course in Bacterial Genetics, A Laboratory Manual and Handbook for Escherichia coli and Related Bacteria”, Cold Spring Harbor Laboratory Press, 1992).

The long DNA fragments cloned with the aid of cosmids or other λ vectors may then be subcloned into suitable vectors commonly used for DNA sequencing. Methods for DNA sequencing are described inter alia in Sanger et al. (Proceedings of the National Academy of Sciences of the United States of America USA, 74:5463-5467, 1977).

The resulting DNA sequences may then be investigated using well-known algorithms or sequence-analysis programs such as the program by Staden (Nucleic Acids Research 14, 217-232(1986)), the program by Marck (Nucleic Acids Research 16, 1829-1836 (1988)) or the GCG program by Butler (Methods of Biochemical Analysis 39, 74-97 (1998)).

In that manner the novel DNA sequence from C. glutamicum that codes for the lipB gene (SEQ ID No. 1) has been obtained and forms part of this invention. Furthermore, the amino acid sequence of the corresponding protein was derived from the available DNA sequence using the method described above. SEQ ID No. 2 represents the amino acid sequence of the resulting lipB gene product.

Coding DNA sequences that are produced from SEQ ID No.1 by degeneracy of the genetic code also form part of the invention. In the same way, DNA sequences that hybridize with SEQ ID No. 1 or parts of SEQ ID No. 1 form part of the invention. Furthermore, to a person skilled in the art, conservative amino acid exchanges, such as replacement of glycine by alanine or of aspartic acid by glutamic acid, in proteins are known as “sense mutations.” These mutations may not lead to any fundamental change in the activity of the protein, i.e. they are functionally neutral mutations. It is also known that changes at the N- and/or C-terminus of a protein do not substantially impair, and may even stabilize, its function. A person skilled in the art will find relevant information inter alia in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in well-known textbooks on genetics and molecular biology. Amino acid sequences that are produced in a corresponding manner from SEQ ID No. 2 also form part of the invention.

Finally, DNA sequences that are prepared from SEQ ID No.1 by the polymerase chain reaction (PCR) using primers that result from SEQ ID No.1 form part of the invention. Such oligonucleotides typically have a length of at least 15 nucleotides.

A person skilled in the art will find instructions for identifying DNA sequences by means of hybridization inter alia in the manual “The DIG System Users Guide for Filter Hybridization” produced by Boehringer Mannheim GmbH (Mannheim, Germany, 1993) and in Liebl et al. (International Journal of Systematic Bacteriology 41: 255-260 (1991)). Hybridization takes place under stringent conditions, that is to say the only hybrids formed are those in which probe and target sequence, i.e. the polynucleotides treated with the probe, are at least 70% identical. It is known that the stringency of hybridization, including the washing step, is influenced or determined by varying the buffer composition, the temperature, and the salt concentration. For reasons explained infra, the hybridization reaction is preferably performed at relatively low stringency as compared with the washing step (Hybaid Hybridisation Guide, Hybaid Limited, Teddington, UK, 1996).

A 5×SSC buffer at a temperature of about 50-68° C. can be used for the hybridization reaction. Probes may also hybridize with polynucleotides that are less than 70% identical to the sequence of the probe. Such hybrids are less stable and are removed by washing under stringent conditions. This can be achieved, for example, by lowering the salt concentration to 2×SSC and optionally then to 0.5×SSC (The DIG System User's Guide for Filter Hybridization, Boehringer Mannheim, Mannheim, Germany, 1995), wherein the adjustments are performed at a temperature of about 50-68° C. By a stepwise increase in the hybridization temperature from 50 to 68° C. in increments of about 1-2° C., polynucleotide fragments can be isolated that are, for example, at least 70% or at least 80% or at least 90% to 95% identical to the sequence of the probe used. Commercial kits containing further instructions for hybridizing are readily obtainable on the market (e.g. DIG Easy Hyb from Roche Diagnostics GmbH, Mannheim, Germany, Catalog No. 1603558).

A person skilled in the art will find instructions for amplifying DNA sequences with the aid of the polymerase chain reaction (PCR) inter alia in the manual by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

During work on the present invention, it was found that Coryneform bacteria produce amino acids, in particular L-lysine, in an improved manner after attenuating the lipB gene.

To achieve attenuation, either the expression of the lipB gene or the catalytic properties of the enzyme protein may be reduced or excluded. Optionally, both measures can be combined.

The reduction in gene expression may be effected by performing the culturing in a suitable manner or by genetic modification (mutation) of the signal structures for gene expression. Signal structures for gene expression are, for example, repressor genes, activator genes, operators, promoters, attenuators, ribosome bonding positions, the start codon, and terminators. A person skilled in the art will find information on this in patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Patek et al. (Microbiology 142: 1297 (1996)), Vasicova et al. (Journal of Bacteriology 181: 6188 (1999)) and in well-known textbooks on genetics and molecular biology such as the textbook by Knippers (“Molekulare Genetik”, 6th edition, Georg Thieme Verlag, Stuttgart, Germany, 1995) or the textbook by Winnacker (“Gene und Klone”, VCH Verlagsgesellschaft, Weinheim, Germany, 1990).

Mutations that lead to a change or reduction in the catalytic properties of enzyme proteins are known from the prior art; examples that may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Mockel (“Die Threonindehydratase aus Corynebacterium glutamicum: Aufhebung der allosterischen Regulation und Struktur des Enzyms”, reports of the Jülich Research Centre, Jül-2906, ISSN09442952, Jülich, Germany, 1994). Summaries found in well-known textbooks on genetics and molecular biology such as e.g. the book by Hagemann (“Allgemeine Genetik”, Gustav Fischer Verlag, Stuttgart, 1986).

These mutations may be transitions, transversions, insertions and deletions. Depending on the effect of amino acid exchange on the enzyme activity, missense mutations or nonsense mutations are referred to. Insertions or deletions of at least one base pair (bp) in a gene lead to frame shift mutations, as a consequence incorrect amino acids are incorporated or translation is terminated prematurely. Deletions of several codons typically lead to complete loss of enzyme activity. Instructions for producing these types of mutations are part of the prior art and may be found in well-known textbooks on genetics and molecular biology such as the textbook by Knippers (“Molekulare Genetik”, 6th edition, Georg Thieme Verlag, Stuttgart, Germany, 1995), the book by Winnacker (“Gene und Klone”, VCH Verlagsgesellschaft, Weinheim, Germany, 1990) or the book by Hagemann (“Allgemeine Genetik”, Gustav Fischer Verlag, Stuttgart, 1986).

A common method of mutating genes in C. glutamicum is the method of gene disruption and gene replacement described by Schwarzer and Puhler (Bio/Technology 9, 84-87 (1991)).

In the gene disruption method, a central part of the coding region of the gene of interest is cloned in a plasmid vector which can replicate in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Netherlands; Bernard et al., Journal of Molecular Biology, 234: 534-541 (1993)) or pEM1 (Schrumpf et al, 1991, Journal of Bacteriology 173:4510-4516). The plasmid vector containing the central part of the coding region of the gene is then transferred into the desired strain of C. glutamicum by conjugation or transformation. The method of conjugation is described, for example, in Schäfer et al. (Applied and Environmental Microbiology 60, 756-759 (1994)). Methods for transformation are described by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over event, the coding region of the gene in question is disrupted by the vector sequence and two incomplete alleles are obtained, on lacking the 3′-and one lacking the 5′-ends. This method was used by Fitzpatrick et al. (Applied Microbiology and Biotechnology 42, 575-580 (1994)) to exclude the recA gene in C. glutamicum.

In the gene replacement method, a mutation such as a deletion, insertion, or base replacement is produced in vitro in the gene of interest. The allele prepared is in turn cloned in a vector that is not replicated in C. glutamicum and this is then transferred into the desired host for C. glutamicum by transformation or conjugation. After homologous recombination by means of a first cross-over event effecting integration and by means of a suitable second cross-over event effecting an excision in the target gene or in the target sequence, incorporation of the mutation or the allele is achieved. This method was used by Peters-Wendisch et al.(Microbiology 144, 915-927 (1998)) to exclude the pyc gene in C. glutamicum by means of a deletion.

A deletion, insertion or base replacement can be incorporated in the lipB gene in this way.

In addition, it may be advantageous for the production of L-amino acids, in particular L-lysine, in addition to attenuation of the lipB gene, to amplify, in particular overexpress, glycolysis, anaploretic processes, the citric acid cycle, the pentose-phosphate cycle, amino acid export and optionally regulatory proteins.

Thus, for example, for the production of L-lysine, one or more genes chosen from the group

the dapA gene coding for dihydrodipicolinate synthase (EP-B 0 197 335),

the gap gene coding for glyceraldehyde-3-phosphate dehydrogenase (Eikmanns (1992), Journal of Bacteriology 174:6076-6086),

the tpi gene coding for triosephosphate isomerase (Eikmanns (1992), Journal of Bacteriology 174:6076-6086),

the pgk gene coding for 3-phosphoglycerate kinase (Eikmanns (1992), Journal of Bacteriology 174:6076-6086),

the zwf gene coding for glucose-6-phosphate dehydrogenase (JP-A-09224661),

the pyc gene coding for pyruvate carboxylase (DE-A-198 31 609),

the lysC gene coding for a feed-back resistant aspartate kinase (Kalinowski et al. (1990), Molecular and General Genetics 224, 317-324; Accession No.P26512), or

the mqo gene coding for malate-quinone-oxidoreductase (Molenaar et al., European Journal of Biochemistry 254, 395-403 (1998)), or

the lysE gene coding for lysine export (DE-A-195 48 222)

the zwal coding for Zwal protein (DE: 199 59 328.0, DSM 13115) may simultaneously be enhanced, in particular over-expressed.

As a result of enhancement, in particular over-expression, the activity or concentration of the corresponding protein is increased, in general, preferably ranging from at least 10%, 25%, 50%, 75%, 100%, 150%, 200%, 300%, 400%, or 500%, up to 1000% or 2000% of the wild-type protein activity or concentration present in the microorganism.

It may also be advantageous for the production of amino acids, in particular L-lysine, in addition to the attenuation of the lipB gene, to simultaneously attenuate one or more genes chosen from the group

the pck gene coding for phosphoenolpyruvate carboxykinase (DE 199 50 409.1, DSM 13047),

the pgi gene coding for glucose-6-phosphate isomerase (U.S. Ser. No. 09/396,478, DSM 12969),

the poxB gene coding for pyruvate oxidase (DE:1995 1975.7, DSM 13114)

the zwa2 gene coding for Zwa2 protein (DE: 199 59 327.2, DSM 13113).

Furthermore, it may be advantageous for the production of amino acids, particularly L-lysine, in addition to attenuation of the lipB gene, to eliminate undesired side reactions (Nakayama: “Breeding of Amino Acid Producing Microorganisms”, in: Overproduction of Microbial Products, Krumphanzl, Sikyta, Vanek (eds.), Academic Press, London, UK, 1982).

Microorganisms prepared according to the invention, for the purposes of producing L-amino acids, in particular L-lysine, may be cultured continuously or batchwise in a batch process, fed batch process, or repeated fed batch process. A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

A suitable culture medium must be used to meet the requirements of the particular strain. Descriptions of culture media for various microorganisms contained in the handbook “Manual of Methods for General Bacteriology” by the American Society for Bacteriology (Washington D.C., USA, 1981).

The carbon sources may be sugars and carbohydrates (e.g., glucose, saccharose, lactose, fructose, maltose, molasses, starch and cellulose), oils and fats (e.g., soya oil, sunflower oil, peanut oil and coconut oil), fatty acids (e.g., palmitic acid, stearic acid and linoleic acid), alcohols (e.g., glycerine and ethanol), and organic acids (e.g., acetic acid). These substances may be used individually or as a mixture.

The nitrogen sources may be organic nitrogen-containing compounds (e.g., peptones, yeast extract, meat extract, malt extract, maize steep liquor, soya bean meal and urea) or inorganic compounds (e.g., ammonium sulfate, ammonium chloride, ammonium phosphate, ammonium carbonate and ammonium nitrate). The sources of nitrogen may be used individually or as a mixture.

The phosphorus sources may be phosphoric acid, potassium dihydrogen phosphate or dipotassium hydrogen phosphate (or the corresponding sodium-containing salts).

Furthermore, the culture medium must contain salts of metals (e.g., magnesium sulfate or iron sulfate) that are required for growth. Finally, essential growth-promoting substances such as amino acids and vitamins may be used in addition to the above-mentioned substances. Moreover, suitable precursors may be added to the culture medium. The starting substances mentioned may be added to the culture in the form of a single batch or may be fed in a suitable manner during fermentation.

Regulation of the pH of the culture may be achieved by addition of basic compounds (e.g., sodium hydroxide, potassium hydroxide, ammonia or ammoniacal liquor) or acidic compounds (e.g., phosphoric acid or sulfuric acid) in a suitable manner. Fatty acid polyglycol esters may be used to control the development of foam. In order to maintain the stability of plasmids, suitable substances having a selective action, such as antibiotics, may be added to the medium. In order to maintain aerobic conditions, oxygen or oxygen-containing gas mixtures, such as air, are passed into the culture. The temperature of the culture is normally 20° C. to 45° C. and is preferably 25° C. to 40° C. Fermentation is continued until the maximum of the desired product has been formed. This objective is normally achieved within 10 hours to 160 hours.

Methods for the determination of L-amino acids are known from the prior art. Analysis may be performed, for example, by anion exchange chromatography followed by ninhydrin derivation as described by Spackman et al. (Analytical Chemistry, 30, (1958), 1190) or it may be performed by reversed phase HPLC as described by Lindroth et al. (Analytical Chemistry (1979) 51: 1167-1174).

The isolation of plasmid DNA from Escherichia coli and all techniques of restriction, Klenow treatment and alkaline phosphatase treatment were performed as described by Sambrook et al. (Molecular Cloning. A Laboratory Manual, 1989, Cold Spring Harbor Laboratory Press, Cold Spring Harbor, N.Y., USA). Methods for the transformation of Escherichia coli and the composition of commonly used nutrient media, such as LB or TY medium, are also described in this handbook.

Having generally described this invention, a further understanding can be obtained by reference to certain specific examples, which are provided herein for purposes of illustration only, and are not intended to be limiting unless otherwise specified.

### EXAMPLES

### Example 1

Production of a Genomic Cosmid Gene Library from C. glutamicum ATCC 13032

Chromosomal DNA from C. glutamicum ATCC 13032 was isolated as described in Tauch et al., (1995, Plasmid 33:168-179), and partly cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Molecular Biochemicals, Mannheim, Germany, product description SAP, Code no. 1758250). The DNA in the cosmid vector SuperCos1 (Wahl et al. (1987), Proceedings of the National Academy of Sciences, USA 84:2160-2164), purchased from Stratagene (La Jolla, USA, product description SuperCos1 Cosmid Vektor Kit, Code no. 251301) was cleaved with the restriction enzyme XbaI (Amersham Pharmacia, Freiburg, Germany, product description XbaI, Code no. 27-0948-02) and also dephosphorylated with shrimp alkaline phosphatase.

The cosmid DNA was cleaved with the restriction enzyme BamHI (Amersham Pharmacia, Freiburg, Germany, product description BamHI, Code no. 27-0868-04). The cosmid DNA so treated was mixed with the treated ATCC13032 DNA and the mixture was additionally treated with T4 DNA ligase (Amersham Pharmacia, Freiburg, Germany, product description T4-DNA-Ligase, Code no.27-0870-04). The ligation mixture was then packed into phages with the aid of Gigapack II XL Packing Extracts (Stratagene, La Jolla, USA, product description Gigapack II XL Packing Extract, Code no. 200217).

For infection of the E. coli strain NM554 (Raleigh et al. 1988, Nucleic Acid Res. 16:1563-1575), the cells were taken up in 10 mM MgSO₄ and mixed with an aliquot of the phage suspension. Infection and titering of the cosmid library were performed as described in Sambrook et al. (1989, Molecular Cloning: A Laboratory Manual, Cold Spring Harbor), wherein the cells were plated out on LB agar (Lennox, 1955, Virology, 1:190)+100 μg/ml ampicillin. After incubation overnight at 37° C., recombinant individual clones were selected.

### Example 2

Isolating and Sequencing the lipB Gene

The cosmid DNA from an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Molecular Biochemicals, Mannheim, Germany, product description SAP, Product No. 1758250). After gel electrophoretic separation, isolation of the cosmid fragments in the size range 1500 to 2000 bp was performed with QiaExII Gel Extraction Kit (Product No. 20021, Qiagen, Hilden, Germany).

The DNA in sequencing vector pZero-I purchased from Invitrogen (Groningen, Netherlands, product description Zero Background Cloning Kit, Product No. K2500-01) was cleaved with the restriction enzyme BamHI (Amersham Pharmacia, Freiburg, Germany, product description BamHI, Product No. 27-0868-04). Ligation of the cosmid fragments in sequencing vector pZero-1 was performed as described in Sambrook et al. (1989, Molecular Cloning: A Laboratory Manual, Cold Spring Harbor), wherein the DNA mixture was incubated overnight with T4 ligase (Pharmacia Biotech, Freiburg, Germany). This ligation mixture was then electroporated in E. coli strain DH5 MCR (Grant, 1990, Proceedings of the National Academy of Sciences, U.S.A., 87:4645-4649) (Tauch et al. 1994, FEMS Microbiol Letters, 123:343-7) and plated out on LB agar (Lennox, 1955, Virology, 1:190) with 50 μg/ml zeocin.

Plasmid preparation of recombinant clones was performed with Biorobot 9600 (Product No. 900200, Qiagen, Hilden, Germany). DNA sequencing was performed using the dideoxy chain termination method according to Sanger et al. (1977, Proceedings of the National Academies of Sciences, U.S.A., 74:5463-5467) with modifications by Zimmermann et al. (1990, Nucleic Acids Research, 18:1067). The “RR dRhodamin Terminator Cycle Sequencing Kit” from PE Applied Biosystems(Product No. 403044, Weiterstadt, Germany) was used. Gel electrophoretic separation and analysis of the sequencing reaction was performed in a “Rotiphorese NF Acrylamid/Bisacrylamid” Gel (29:1) (Product No. A124.1, Roth, Karlsruhe, Germany) using the “ABI Prism 377” sequencing instrument from PE Applied Biosystems (Weiterstadt, Germany).

The raw sequencing data obtained were then processed using the Staden program package (1986, Nucleic Acids Research, 14:217-231) Version 97-0. The individual sequences of the pZero1 derivatives were assembled to give a cohesive contig. Computer aided coding region analyses were drawn up with the program XNIP (Staden, 1986, Nucleic Acids Research, 14:217-231). Further analyses were performed using the “BLAST search programs” (Altschul et al., 1997, Nucleic Acids Research, 25:33893402) against the non-redundant database of the “National Center for Biotechnology Information” (NCBI, Bethesda, Md., USA).

The resulting nucleotide sequence obtained is shown in SEQ ID No. 1. Analysis of the nucleotide sequence revealed an open reading frame of 804 bp, which was designated the lipB gene. The lipB gene coded for a polypeptide of 269 amino acids.

Obviously, numerous modifications and variations on the present invention are possible in light of the above teachings. It is therefore to be understood that within the scope of the appended claims, the invention may be practiced otherwise than as specifically described herein.

What is claimed is:

1. An isolated polynucleotide, which encodes a protein comprising the amino acid sequence of SEQ ID NO:2.

2. The isolated polynucleotide of claim 1, wherein said protein has lipoprotein ligase B activity.

3. A vector comprising the isolated polynucleotide of claim 1.

4. A host cell comprising the isolated polynucleotide of claim 1.

5. The host cell of claim 4, which is a Coryneform bacterium.

6. The host cell of claim 4, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

7. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

8. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.

9. A process for screening for polynucleotides, which encode a protein lipoprotein ligase B activity comprising
a) hybridizing the isolated polynucleotide of claim 1 to the polynucleotide to be screened;
b) expressing the polynucleotide to produce a protein; and
c) detecting the presence or absence of lipoprotein ligase B activity in said protein.

10. A method for making lipoprotein ligase B protein, comprising
a) culturing the host cell of claim 4 for a duration of time under conditions suitable for expression of lipoprotein ligase B protein; and
b) collecting the lipoprotein ligase B protein.

11. An isolated polynucleotide, which comprises SEQ ID NO:1.

12. An isolated polynucleotide, which is complimentary to the polynucleotide of claim 11.

13. An isolated polynucleotide, which is at least 70% identical to the polynucleotide of claim 11.

14. An isolated polynucleotide, which is at least 80% identical to the polynucleotide of claim 11.

15. An isolated polynucleotide, which is at least 90% identical to the polynucleotide of claim 11.

16. An isolated polynucleotide, which comprises at least 15 consecutive nucleotides of the polynucleotide of claim 11.

17. An isolated polynucleotide, which hybridizes under stringent conditions to the polynucleotide of claim 11; wherein said stringent conditions comprise washing in 5×SSC at a temperature from 50 to 68° C.

18. The isolated polynucleotide of claim 11, which encodes a protein having lipoprotein ligase B activity.

19. A vector comprising the isolated polynucleotide of claim 11.

20. A host cell comprising the isolated polynucleotide of claim 11.

21. The host cell of claim 20, which is a Coryneform bacterium.

22. The host cell of claim 20, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

23. A process for screening for polynucleotides, which encode a protein having lipoprotein ligase B activity comprising
a) hybridizing the isolated polynucleotide of claim 11 to the polynucleotide to be screened;
b) expressing the polynucleotide to produce a protein; and
c) detecting the presence or absence of lipoprotein ligase B activity in said protein.

24. A method for detecting a nucleic acid with at least 70% homology to nucleotide of claim 11, comprising contacting a nucleic acid sample with a probe or primer comprising at least 15 consecutive nucleotides of the nucleotide sequence of claim 11, or at least 15 consecutive nucleotides of the complement thereof.

25. A method for producing a nucleic acid with at least 70% homology to nucleotide of claim 11, comprising contacting a nucleic acid sample with a primer comprising at least 15 consecutive nucleotides of the nucleotide sequence of claim 11, or at least 15 consecutive nucleotides of the complement thereof.

26. A method for making lipoprotein ligase B protein, comprising
a) culturing the host cell of claim 20 for a duration of time under conditions suitable for expression of lipoprotein ligase B protein; and
b) collecting the lipoprotein ligase B protein.

27. Coryneform bacterium, which comprises attenuated expression of the lipB gene.

28. The Coryneform bacterium of claim 27, wherein said lipB gene comprises the polynucleotide sequence of SEQ ID NO:1.

29. A process for producing L-amino acids comprising culturing a bacterial cell in a medium suitable for producing L-amino acids, wherein said bacterial cell comprises attenuated expression of the lipB gene.

30. The process of claim 29, wherein said bacterial cell is a Coryneform bacterium or Brevibacterium.

31. The process of claim 30, wherein said bacterial cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

32. The process of claim 29, wherein said lipB gene comprises the polynucleotide sequence of SEQ ID NO:1.

33. The process of claim 29, wherein said L-amino acid is L-lysine.

34. The process of claim 29, wherein said bacteria further comprises at least one gene whose expression is enhanced, wherein said gene is selected from the group consisting of dapA, gap, tpi, pgk, zwf; pyc, lysC, mqo, lysE, and zwa1.

35. The process of claim 29, wherein said bacteria further comprises at least one gene whose expression is attenuated, wherein said gene is selected from the group consisting of pck, pgi, poxB, and zwa2.

36. An isolated polypeptide comprising the amino acid sequence of SEQ ID NO:2.
